(19) United States
(12) Patent Application Publication
(10) Pub. No.: US 2009/0022666 A1
(43) Pub. Date: Jan. 22, 2009

(54) Methods and systems relating to mitochondrial DNA information

(75) Inventors: Roderick A. Hyde, Redmond, WA (US); Muriel Y. Ishikawa, Livermore, CA (US); Eric C. Leuthardt, St. Louis, MO (US); Dennis J. Rivet, St. Louis, MO (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/880,453

(22) Filed: Jul. 19, 2007

(51) Int. Cl. A61K 49/00; A61P 43/00; C12Q 1/68; G01N 33/50

(52) U.S. Cl. 424/9.1; 435/6; 702/20

(57) ABSTRACT

The present application relates, in general, to a system or a method for detection or treatment. In some aspects, a system includes at least one computer program for use with at least one computer system and wherein the computer program includes a plurality of instructions including but not limited to one or more instructions for making at least one correlation between mitochondrial DNA information regarding at least one person and information regarding at least one clinical trial involving the at least one person.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. ______[To Be Assigned by the USPTO], entitled METHODS AND SYSTEMS RELATING TO EPIGENETIC INFORMATION, naming Roderick A. Hyde; Muriel Y. Ishikawa; Eric C. Leuthardt; Dennis J. Rivet; and Lowell L. Wood, Jr. as inventors, filed contemporaneously herewith, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### SUMMARY

In one aspect, a method includes, but is not limited to, determining at least one correlation between at least one aspect of mitochondrial DNA information regarding at least one individual and information regarding at least one clinical outcome following receipt by the at least one individual of at least one medical therapy. In one aspect, a method includes, but is not limited to, determining a similarity or a dissimilarity in at least one aspect of mitochondrial DNA information regarding the at least one first individual to at least one aspect of mitochondrial information regarding at least one second individual, wherein the at least one second individual attained a clinical outcome following receipt of the at least one medical therapy. In addition to the foregoing, other method aspects are described in the claims, drawings, and text forming a part of the present disclosure.

In one or more various aspects, related systems include but are not limited to circuitry or programming for effecting the herein-referenced method aspects; the circuitry or programming can be virtually any combination of hardware, software, or firmware configured to effect the herein—referenced method aspects depending upon the design choices of the system designer.

In one aspect, a system includes, but is not limited to, at least one computer program for use with at least one computer system and wherein the computer program includes a plurality of instructions, including but not limited to, one or more instructions for determining at least one correlation between at least one aspect of mitochondrial DNA information regarding at least one individual and information regarding at least one clinical outcome following receipt by the at least one individual of at least one medical therapy. In one aspect, a system includes, but is not limited to, at least one computer program for use with at least one computer system and wherein the computer program includes a plurality of instructions, including but not limited to one or more instructions for determining a similarity or a dissimilarity in at least one aspect of mitochondrial DNA information regarding the at least one first individual to at least one aspect of mitochondrial DNA information regarding at least one second individual, wherein the at least one second individual attained a clinical outcome following receipt of the at least one medical therapy. In one aspect, a system includes, but is not limited to, at least one computer program for use with at least one computer system and wherein the computer program includes a plurality of instructions, including but not limited to one or more instructions for making one or more correlations between at least one aspect of mitochondrial DNA information obtained regarding at least one individual and information regarding at least one medical therapy involving the at least one individual; and one or more instructions for applying one or more of the one or more correlations to at least one aspect of mitochondrial DNA information obtained regarding a plurality of people. In addition to the foregoing, other system aspects are described in the claims, drawings, and text forming a part of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 illustrates some aspects of a system that may serve as an illustrative environment for subject matter technologies.

FIG. 2 depicts aspects of a system such as that described in FIG. 1.

FIG. 3 shows aspects of a system such as that described in FIG. 1.

FIG. 4 illustrates aspects of a system such as that described in FIG. 1.

FIG. 5 depicts aspects of a system such as that described in FIG. 1.

FIG. 6 shows aspects of a system such as that described in FIG. 1.

FIG. 7 illustrates aspects of a system such as that described in FIG. 1.

FIG. 8 depicts some aspects of a system that may serve as an illustrative environment for subject matter technologies.

FIG. 9 shows aspects of a system such as that described in FIG. 8.

FIG. 10 illustrates aspects of a system such as that described in FIG. 8.

FIG. 11 shows a logic flowchart of a process.

FIG. 12 illustrates a logic flowchart of a process, such as that depicted in FIG. 11.

FIG. 13 depicts a logic flowchart of a process, such as that depicted in FIG. 11.

FIG. 14 shows a logic flowchart of a process, such as that depicted in FIG. 11.

FIG. 15 illustrates a logic flowchart of a process, such as that depicted in FIG. 11.

FIG. 16 depicts some aspects of a system.

FIG. 17 illustrates aspects of a system, such as that depicted in FIG. 16.

FIG. 18 shows aspects of a system, such as that depicted in FIG. 16.

FIG. 19 depicts aspects of a system, such as that depicted in FIG. 16.

FIG. 20 illustrates a logic flowchart of a process.

FIG. 21 shows a logic flowchart of a process, such as that depicted in FIG. 20.

FIG. 22 depicts a logic flowchart of a process, such as that depicted in FIG. 20.

FIG. 23 illustrates a logic flowchart of a process, such as that depicted in FIG. 20.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present application uses formal outline headings for clarity of presentation. However, it is to be understood that the outline headings are for presentation purposes, and that different types of subject matter may be discussed throughout the application (e.g., device(s)/structure(s) may be described under process(es)/operations heading(s) or process(es)/operations may be discussed under structure(s)/process(es) headings; or descriptions of single topics may span two or more topic headings). Hence, the use of the formal outline headings is not intended to be in any way limiting.

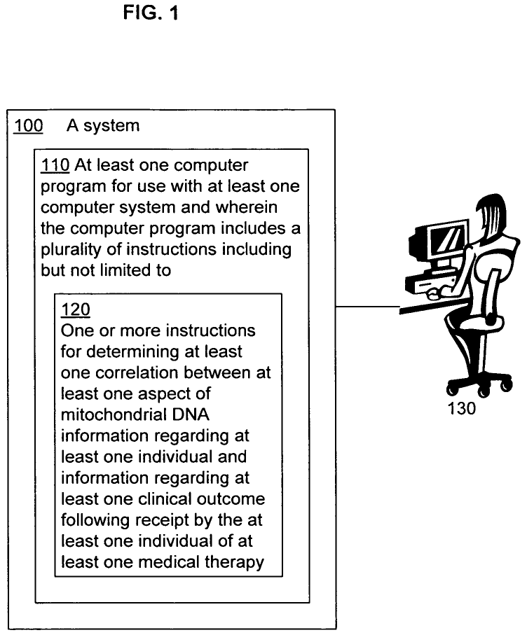

With reference to the figures, and with reference now to FIG. 1, depicted is one aspect of a system that may serve as an illustrative environment of or for subject matter technologies, for example, at least one computer program for use with at least one computer system and wherein the computer program includes a plurality of instructions, including but not limited to, one or more instructions for determining at least one correlation between at least one aspect of mitochondrial DNA information regarding at least one individual and information regarding at least one clinical outcome following receipt by the at least one individual of at least one medical therapy. Accordingly, the present application first describes certain specific example systems of FIG. 1; thereafter, the present application illustrates certain specific example structures and processes. Those having skill in the art will appreciate that the specific structures and processes described herein are intended as merely illustrative of their more general counterparts.

### A. Structure(s) and or System(s)

Continuing to refer to FIG. 1, depicted is a partial view of a system that may serve as an illustrative environment of or for subject matter technologies. One or more users 130 may use a system 100 including at least one computer program 110 for use with at least one computer system, wherein the at least one computer program 110 includes a plurality of instructions. One or more users 130 may include, for example, administrators, medical personnel, pharmacists, geneticists, researchers or technicians. Although a single user is shown in FIG. 1, in some embodiments at least one group of users or at least one series of users may interact with the system. In some embodiments, the one or more users 130 may include a computer system, artificial intelligence system (AI) or other circuitry. The at least one computer program 110 may include one or more instructions for determining at least one correlation between at least one aspect of mitochondrial DNA information regarding at least one individual and information regarding at least one clinical outcome following receipt by the at least one individual of at least one medical therapy 120. A correlation may be established by, for example, statistical methods or by a general relationship between the data sets. Medical therapy may include the administration of a therapeutic agent, or the application of a medical procedure, or device. Medical therapy may include therapies that include at least one energy source, such as, for example, radiation, phototherapy, or ultrasound.

At least one aspect of mitochondrial DNA information as described herein may originate from one or more of a number of sources. Mitochondrial DNA information, for example, may originate from one or more array based system, such as the GeneChip® Human Mitochondrial Resequencing Array 2.0, sold by Affymetrix, with corporate headquarters in Santa Clara Calif. See, e.g., the GeneChip® Human Mitochondrial Resequencing Array 2.0 Data Sheet, which is herein incorporated by reference. Mitochondrial DNA information may, for example, originate with a hybridization-based system, such as the Signet™ Mitochondrial DNA Screening System, sold by Marlingen Biosciences Inc., with corporate headquarters in Ijamsville Md. See, e.g., the Signet™ Mitochondrial DNA Screening System brochure, which is herein incorporated by reference. Mitochondrial DNA information may, for example, originate from one or more high performance liquid chromatography (HPLC) techniques such as described by Bayat et al., Mitochondrial mutation detection using enhanced multiplex denaturing high-performance liquid chromatography, International Journal of Immunogenetics 32: 199-205 (2005), which is herein incorporated by reference. In some embodiments, mitochondrial DNA information may originate with more than one technique or method. See, for example, U.S. Pat. No. 6,967,016 to van Gemen et al., titled “Method of determining therapeutic activity or possible side effects of a medicament”, which is herein incorporated by reference.

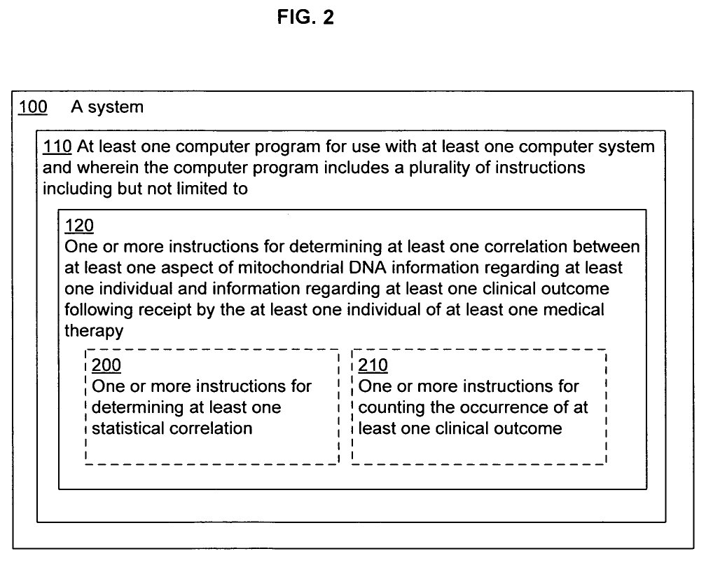

FIG. 2 depicts alternate embodiments of the system of FIG. 1. In some embodiments, the one or more instructions for determining at least one correlation 120 may include one or more instructions for determining at least one statistical correlation 200. In some embodiments, the one or more instructions for determining at least one correlation 120 may include one or more instructions for counting the occurrence of at least one clinical outcome 210. In various aspects, the at least one statistical correlation may include, for example, at least one linear correlation, at least one nonlinear correlation, functional dependency or other mathematical relationship. The at least one statistical correlation may or may not be associated with some type of causality, real or implied, proven or unproven. In some embodiments, the counting the occurrence of at least one clinical outcome may include counting a single occurrence of an outcome, such as, for example, illness, allergic reaction, bleeding, stroke, one or more side effects, or death.

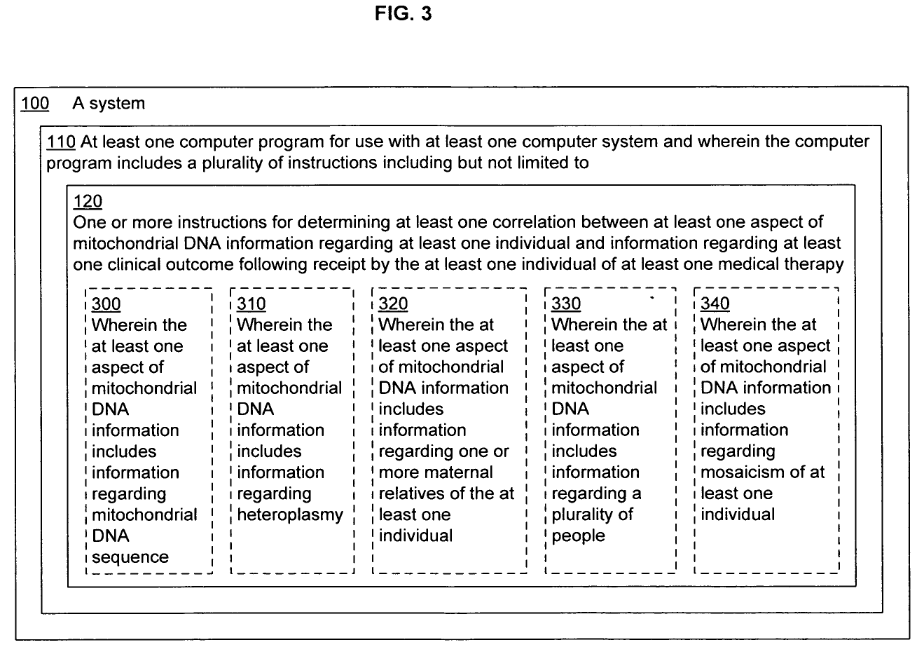

FIG. 3 illustrates alternate embodiments of the system of FIG. 1. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions wherein the at least one aspect of mitochondrial DNA information includes information regarding mitochondrial DNA sequence 300. For example, information regarding mitochondrial DNA sequence may include information regarding linear DNA sequence such as polymorphisms or mutations. See, for example, Ruiz-Pesini et al., An enhanced MITOMAP with a global mtDNA mutational phylogeny, Nucleic Acids Research 35: D823-D828 (2007), which is herein incorporated by reference. Information regarding mitochondrial DNA sequence may include, for example, information regarding the class, group or homology of one or more mitochondrial DNA sequence regions with at least one known mitochondrial DNA class(es), group(s), or sequence(s). See, e.g. Wallace, Mitochondrial DNA sequence variation in human evolution and disease, PNAS USA 91: 8739-8746 (1994) and Khrapko et al., Mitochondrial mutational spectra in human cells and tissues, PNAS USA 94: 13798-13802 (1997), which are herein incorporated by reference. In some embodiments, the one or more instructions for determining at least one correlation 120 may include one or more instructions wherein the at least one aspect of mitochondrial DNA information includes information regarding heteroplasmy 310. Information regarding heteroplasmy, for example, may include information regarding heteroplasmy between tissue types or within tissue types, and may include information regarding heteroplasmy of an individual or multiple individuals. See, for example, Simonetti et al., Accumulation of deletions in human mitochondrial DNA during normal aging: analysis by quantitative PCR, Biochimica et Biophysica Acta 1180: 113-122 (1992), and Liu et al., Mutations in mitochondrial DNA accumulate differentially in three different human tissues during ageing, Nucleic Acids Research 26: 1268-1275 (1998) which are herein incorporated by reference. In some embodiments, one or more instructions for determining at least one correlation 120 include one or more instructions wherein the at least one aspect of mitochondrial DNA information includes information regarding one or more maternal relatives of the at least one individual 320. For example, information regarding one or more maternal relatives of the at least one individual may include information regarding the individual's biological mother, maternal grandmother, maternal aunts and uncles, or maternal cousins. In some embodiments, one or more instructions for making at least one correlation include one or more instructions wherein the at least one aspect of mitochondrial DNA information includes information regarding a plurality of people 330. For example, information regarding a plurality of people may include information regarding at least one biological family unit, at least one ethnic group, at least one geographically-defined population, or at least one clinically-defined population. In some embodiments, one or more instructions for making at least one correlation include one or more instructions wherein the at least one aspect of mitochondrial DNA information includes information regarding mosaicism of at least one individual person 340. The term “mosaicism”, as used herein, denotes, for example, situations where two or more cellular subtypes arise during the lifespan of an organism, situations where two or more cellular subtypes originate with the first cell of an organism and situations where the origin of the cellular subtypes is unclear. The term “mosaicism”, as used herein, may include somatic mosaicism, gonadal mosaicism, or chimerism. For example, information regarding mosaicism of at least one individual person may include information regarding mosaicism of two or more tissue types, or mosaicism within at least one tissue type. For example, information regarding mosaicism of at least one individual person may include information such as the presence or absence of mosaicism, the location of mosaicism, the tissue or tissues involved in the mosaicism, or the proportion of various subtypes of cells in mosaic tissue. For more information regarding somatic mosaicism, see Youssoufian and Pyeritz, Mechanisms and consequences of somatic mosaicism in humans, Nature Reviews Genetics, 3: 748-758 (2002), which is herein incorporated by reference.

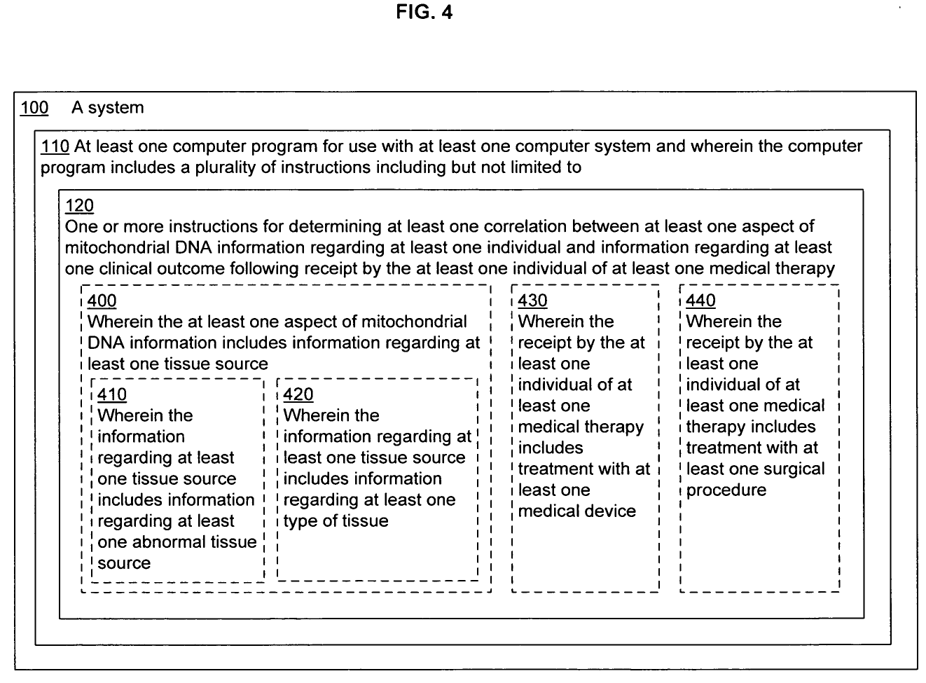

FIG. 4 illustrates alternate embodiments of the system of FIG. 1. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions wherein the at least one aspect of mitochondrial DNA information includes information regarding at least one tissue source 400. For example, the information regarding at least one tissue source may include information regarding at least one tissue type, individual source, or handling of at least one tissue sample. For example, the information regarding at least one tissue source may include information regarding the origin, storage, pathology, pathological subtype, or handling of the tissue. For example, the information regarding at least one tissue source may include information regarding at least one physical, spatial or relative anatomic source. See, e.g., Kujoth et al., The role of mitochondrial DNA mutations in mammalian aging, PLOS Genetics, 3: e24 (2007), which is herein incorporated by reference. In some embodiments, the one or more instructions wherein the at least one aspect of mitochondrial DNA information includes information regarding at least one tissue source 400 may include one or more instructions wherein the information regarding at least one tissue source includes information regarding at least one abnormal tissue source 410. For example, the information regarding at least one abnormal tissue source may include information regarding at least one clinical diagnosis, at least one pathology report, at least one surgical report, handling of at least one tissue sample, or storage of at least one tissue sample. For example, the information regarding at least one abnormal tissue source may include information regarding a neoplastic source, a displastic source, a diseased source, an infectious source or a cancerous source. In some embodiments, the one or more instructions wherein the at least one aspect of mitochondrial DNA information includes information regarding at least one tissue source 400 may include one or more instructions wherein the information regarding at least one tissue source includes information regarding at least one type of tissue 420. For example, information regarding at least one type of tissue may include information regarding at least one clinical diagnosis, at least one pathology report, or at least one surgical report. For example, the information regarding at least one type of tissue may include the origin tissue type, the handling of the tissue, or one or more treatments to the tissue. In some embodiments, the information regarding at least one type of tissue may include cellular developmental stage, lineage, or status. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions wherein the information regarding at least one clinical trial includes at least one parameter which is or is not planned in advance of one or more of the at least one clinical trial. For example, the at least one parameter that is planned in advance may include disease status, infection status, gender, height, weight, or clinical diagnosis. For example, at least one parameter that is planned in advance may include age, geographic location and other demographic variables. For example, the at least one parameter that is not planned in advance may include allergic reactions, side effects, interaction with at least one medical treatment, or death. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions wherein the receipt by the at least one individual of at least one medical therapy includes treatment with at least one medical device 430. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions wherein the receipt by the at least one individual of at least one medical therapy includes treatment with at least one surgical procedure 440.

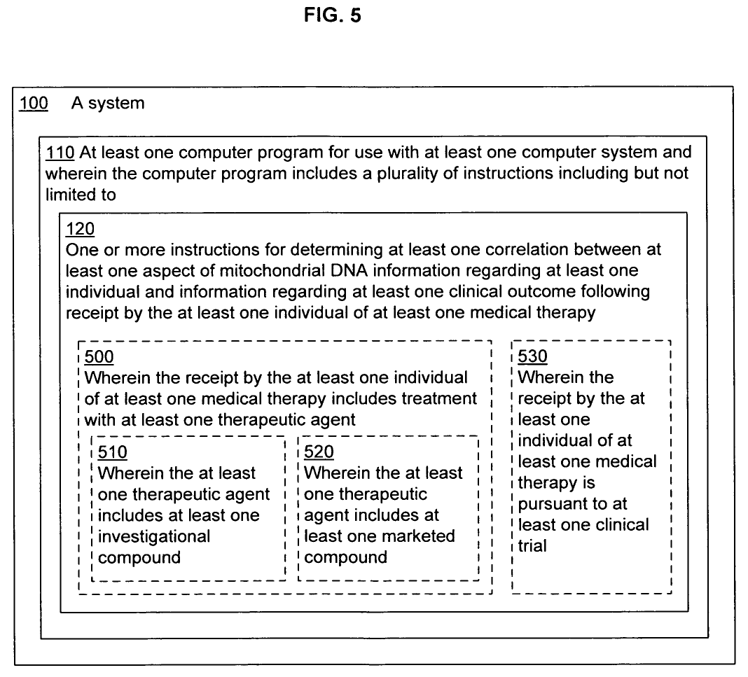

FIG. 5 illustrates alternate embodiments of the system of FIG. 1. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions wherein the receipt by the at least one individual of at least one medical therapy includes treatment with at least one therapeutic agent 500. For example, some embodiments may include at least one therapeutic agent, wherein the at least one therapeutic agent may for example, include at least one drug, biologic, biological material, formulation, pharmaceutical, nutraceutical, dietary supplement, vitamin or compound. A therapeutic agent may be administered, for example, in a variety of ways including oral, topical, iv, ip, sc, intranasal, and inhalation. In some embodiments, the one or more instructions wherein receipt by the at least one individual of at least one medical therapy includes treatment with at least one therapeutic agent 500 may include one or more instructions wherein the at least one therapeutic agent includes at least one investigational compound 510. As used herein, the term “investigational compound” means an experimental active ingredient that is intended to furnish, for example, pharmacological activity or other direct effect in the diagnosis, cure, mitigation, treatment, or prevention of disease or to affect the structure or any function of the human body. For example, the at least one investigational compound may include at least one novel compound, biologic, biological material, drug or formulation, or may include methods or regimens of treatment. As further example, the at least one investigational compound may include a previously known, or marketed, compound, drug or formulation being used in an investigational manner. In some embodiments, the one or more instructions wherein receipt by the at least one individual of at least one medical therapy includes treatment with at least one therapeutic agent 500 may include one or more instructions wherein the at least one therapeutic agent includes at least one marketed compound 520. As used herein, “marketed compound” includes at least one compound, drug or formulation that is commercially available, privately available, or attainable through collaborative agreements. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions wherein the receipt by the at least one individual of at least one medical therapy is pursuant to at least one clinical trial 530.

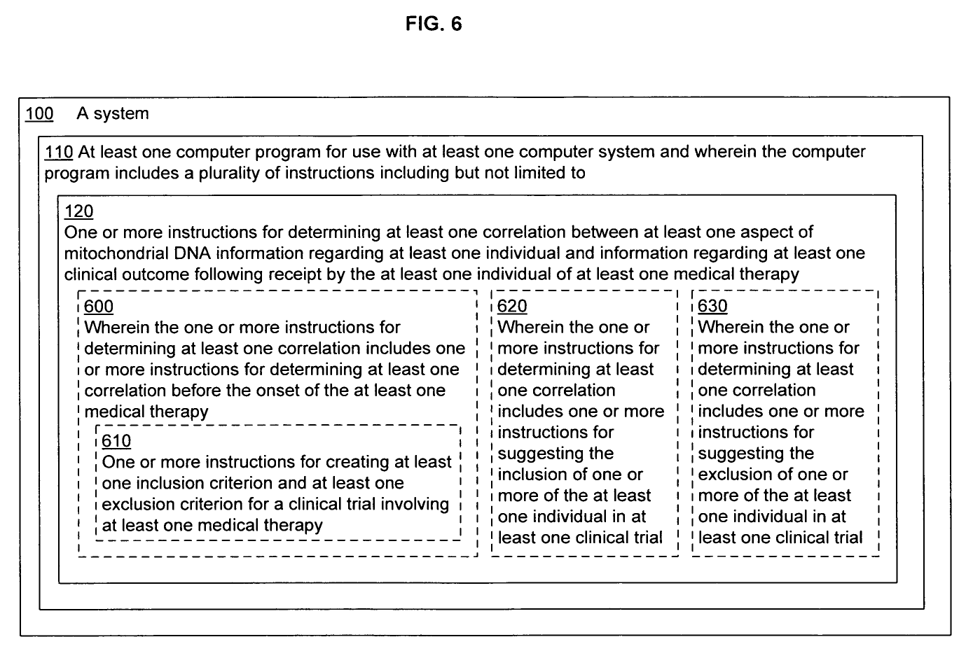

FIG. 6 illustrates some possible aspects of the system depicted in FIG. 1. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions for determining at least one correlation before the onset of the at least one medical therapy 600. In some embodiments, the one or more instructions for determining at least one correlation before the onset of the at least one medical therapy 600 may include one or more instructions for creating at least one inclusion criterion and at least one exclusion criterion for a clinical trial involving at least one medical therapy 610. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions wherein the one or more instructions for determining at least one correlation includes one or more instructions for suggesting the inclusion of one or more of the at least one individual in at least one clinical trial 620. In some embodiments, the one or more instructions for determining at least one correlation between at least one aspect of mitochondrial DNA information regarding at least one individual and information regarding at least one clinical outcome following receipt by the at least one individual of at least one medical therapy 120 may include one or more instructions wherein the one or more instructions for determining at least one correlation includes one or more instructions for suggesting the exclusion of one or more of the at least one individual in at least one clinical trial 630.

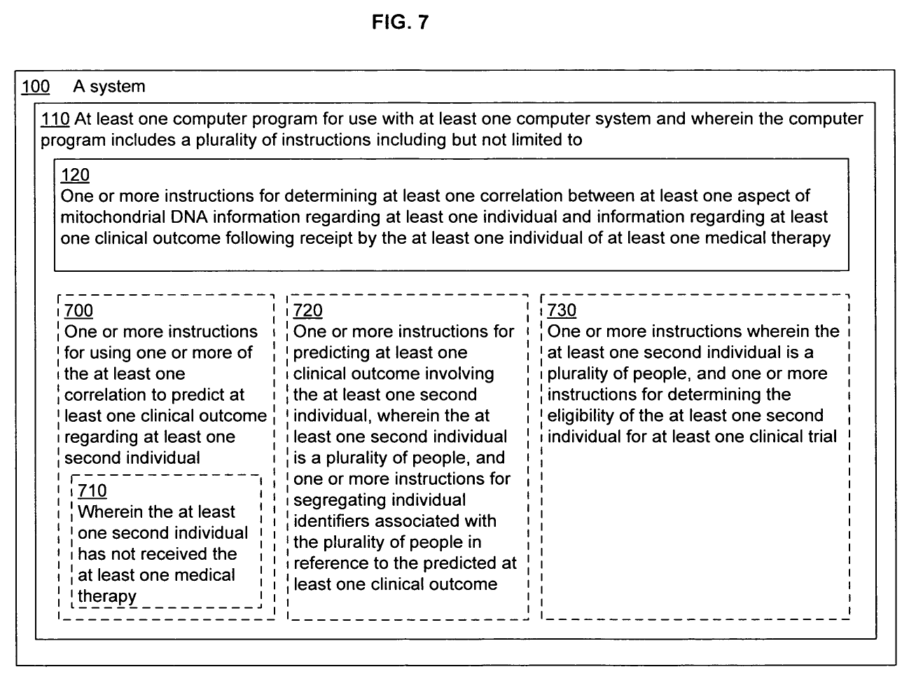

FIG. 7 illustrates some possible aspects of the system depicted in FIG. 1. Some embodiments may include one or more instructions for using one or more of the at least one correlation to predict at least one clinical outcome regarding at least one second individual 700. In some embodiments, the one or more instructions for using one or more of the at least one correlation to predict at least one clinical outcome regarding at least one second individual 700 may include one or more instructions wherein the at least one second individual has not received the at least one medical therapy 710. In some embodiments, the one or more instructions for using one or more of the at least one correlation to predict at least one clinical outcome regarding at least one second individual 700 may include one or more instructions for predicting at least one clinical outcome involving the at least one second individual, wherein the at least one second individual is a plurality of people, and one or more instructions for segregating individual identifiers associated with the plurality of people in reference to the predicted at least one clinical outcome 720. In some embodiments, the instructions for predicting at least one clinical outcome involving one or more of the at least one second individual may include one or more instructions for predicting at least one positive, negative, or no effect clinical outcome. At least one positive clinical outcome may include attaining a clinical endpoint of a clinical trial, or attaining a beneficial effect whether or not a part of a formal clinical endpoint of a clinical trial. Such beneficial effects may include but are not limited to, alleviation of symptoms, alleviation of clinical indicators of disease, increased comfort of the at least one second individual, drug tolerability, survival, increased time to progression of disease, or regression of disease. At least one negative clinical outcome may include any adverse effect, including but not limited to, failure to attain a clinical endpoint of a clinical trial, or failing to attain a beneficial effect, for example, toxicity, illness, allergic reaction, bleeding, stroke, progression of disease, one or more side effects, or death. Alternatively, a clinical outcome may include the measurement of at least one biochemical, biological or physiological parameter such as, by way of non-limiting example, viral load, infection level, blood cell count, cytokine level, drug concentration, drug pharmacokinetic profile, or drug absorption. In some embodiments, the instructions for predicting at least one clinical outcome may include one or more instructions for predicting at least one mitochondrial DNA outcome, such as, for example, a relative reduction or increase in one or more species of mitochondrial DNA within one or more cells. One of skill in the art will readily recognize that the above outcomes are illustrative and myriad other positive, negative, or no effect outcomes are possible and selection of such may be dependent on the design and structure of a particular study and the desired results. In some embodiments, the one or more instructions for using one or more of the at least one correlation to predict at least one clinical outcome regarding at least one second individual 700 may include one or more instructions wherein the at least one second person is a plurality of people, and one or more instructions for determining the eligibility of the at least one second individual for at least one clinical trial 730.

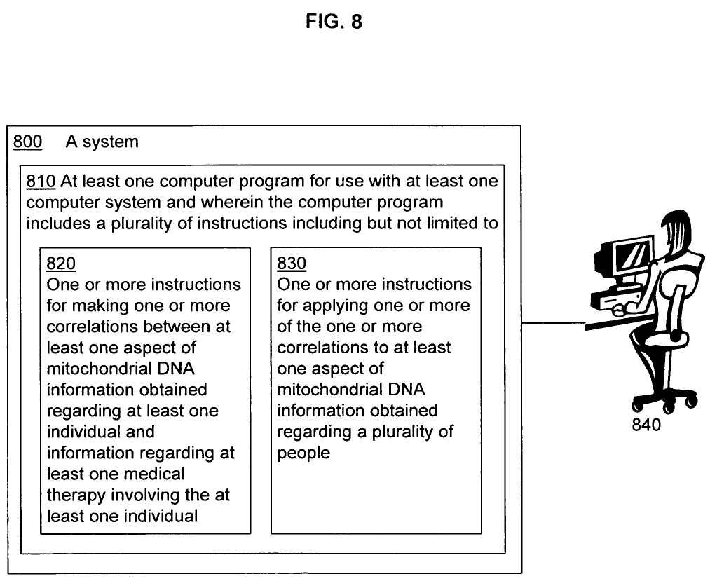

FIG. 8 depicts a partial view of a system that may serve as an illustrative environment of or for subject matter technologies. One or more users 840 may use a system 800 including at least one computer program 810 for use with at least one computer system, wherein the at least one computer program 810 includes a plurality of instructions. One or more users 840 may include, for example, administrators, medical personnel, pharmacists, geneticists, researchers or technicians. Although a single user is shown in FIG. 8, in some embodiments at least one group of users or at least one series of users may interact with the system. In some embodiments, the one or more users 840 may include a computer system, artificial intelligence system (AI) or other circuitry. The at least one computer program 810 may include one or more instructions for making one or more correlations between at least one aspect of mitochondrial DNA information obtained regarding at least one individual and information regarding at least one medical therapy involving the at least one individual 820. The at least one computer program 810 may include one or more instructions for applying one or more of the one or more correlations to at least one aspect of mitochondrial DNA information obtained regarding a plurality of people 830.

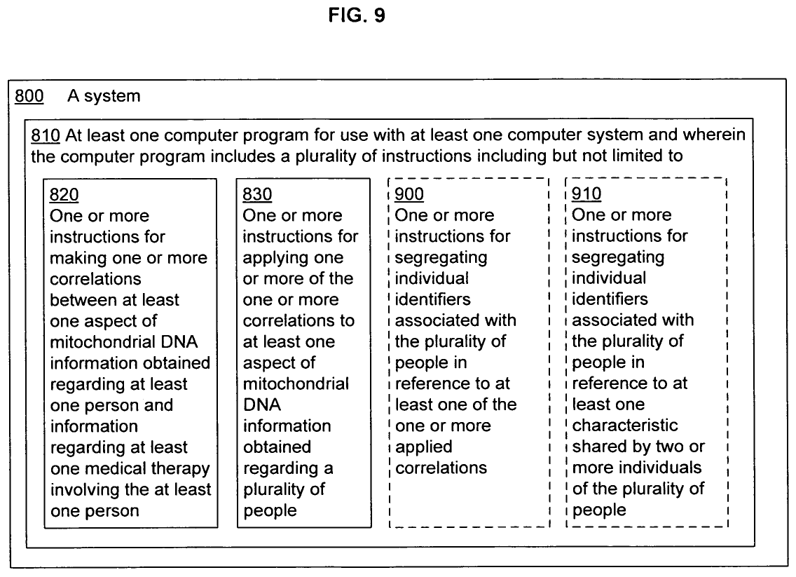

FIG. 9 illustrates aspects of a system such as that depicted in FIG. 8. In some embodiments, the at least one computer program 810 may include one or more instructions for segregating individual identifiers associated with the plurality of people in reference to at least one of the one or more applied correlations 900. In some embodiments, the at least one computer program 810 may include one or more instructions for segregating individual identifiers associated with the plurality of people in reference to at least one characteristic shared by two or more individuals of the plurality of people 910.

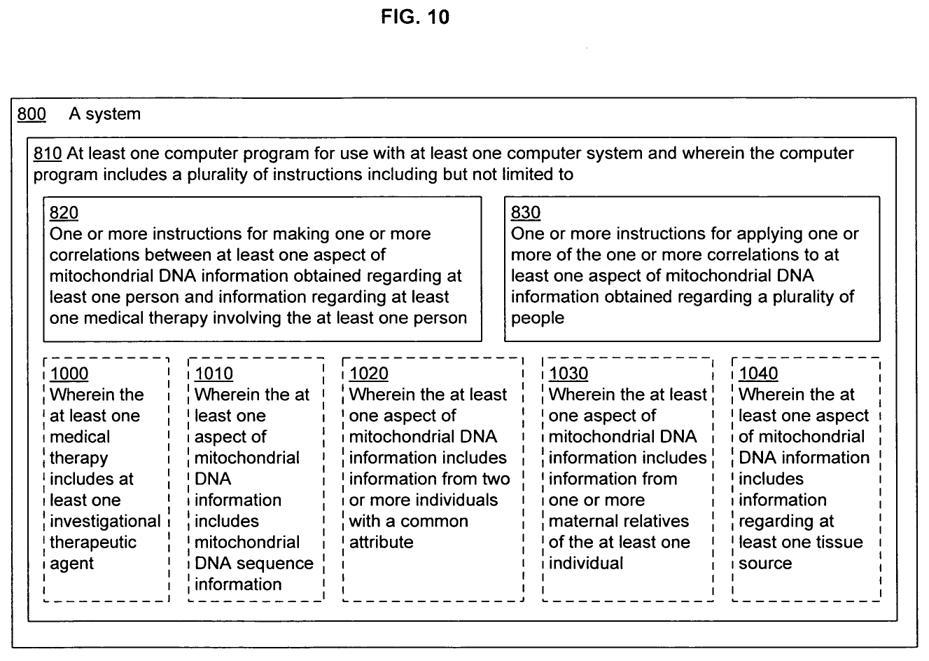

FIG. 10 illustrates aspects of a system such as that depicted in FIG. 8. In some embodiments, the at least one computer program 810 may include one or more instructions wherein the at least one medical therapy includes at least one investigational therapeutic agent 1000. For example, the at least one investigational therapeutic agent may include at least one compound, pharmaceutical, nutraceutical, vitamin, dietary supplement, drug, or formulation. As further example, the at least one investigational therapeutic agent may include a previously known compound, pharmaceutical, nutraceutical, vitamin, dietary supplement, drug, or formulation being used in an investigational manner. In some embodiments, the at least one computer program 810 may include one or more instructions wherein the at least one aspect of mitochondrial DNA information includes mitochondrial DNA sequence information 1010. For example, mitochondrial DNA sequence information may include information regarding linear DNA sequence such as polymorphisms or mutations. Mitochondrial DNA sequence information may include, for example, information regarding the class, group or homology of one or more mitochondrial DNA sequence regions with at least one known mitochondrial DNA class(es), group(s), or sequence(s). In some embodiments, the at least one computer program 810 may include one or more instructions wherein the at least one aspect of mitochondrial DNA information includes information from two or more people with a common attribute 1020. For example, the at least one aspect of mitochondrial DNA information may include information regarding two or more people with a common attribute such as gender, height, weight, diabetes status, heart disease status, medical diagnosis, results on one or more medical tests, or ethnic background. For example, a common attribute may include environmental attributes, such as exposure to a pathogen, a teratogen, a chemical substance. In some embodiments, the at least one computer program 810 may include one or more instructions wherein the at least one aspect of mitochondrial DNA information includes information from one or more maternal relatives of the at least one individual 1030. For example, information from one or more maternal relatives of the at least one individual person may include information from the biological mother, maternal grandmother, maternal aunts and uncles, or maternal cousins of the at least one individual person. In some embodiments, the at least one computer program 810 may include one or more instructions wherein the at least one aspect of mitochondrial DNA information includes information regarding at least one tissue source 1040. For example, the information regarding at least one tissue source may include information regarding the origin, storage, pathology, or handling of the tissue.

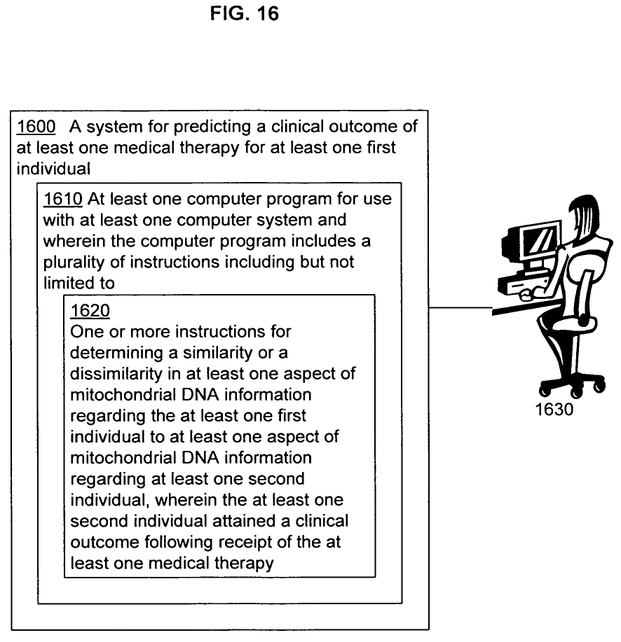

FIG. 16 depicts a partial view of a system that may serve as an illustrative environment of or for subject matter technologies. One or more users 1630 may use a system for predicting a clinical outcome of at least one medical therapy for at least one first individual 1600 including at least one computer program 1610 for use with at least one computer system, wherein the at least one computer program 1610 includes a plurality of instructions. One or more users 1630 may include, for example, administrators, medical personnel, pharmacists, geneticists, researchers or technicians. Although a single user is shown in FIG. 16, in some embodiments at least one group of users or at least one series of users may interact with the system. In some embodiments, the one or more users 1630 may include a computer system, artificial intelligence system (AI) or other circuitry. The at least one computer program 1610 may include one or more instructions for determining a similarity or a dissimilarity in at least one aspect of mitochondrial DNA information regarding the at least one first individual to at least one aspect of mitochondrial DNA information regarding at least one second individual, wherein the at least one second individual attained a clinical outcome following receipt of the at least one medical therapy 1620.

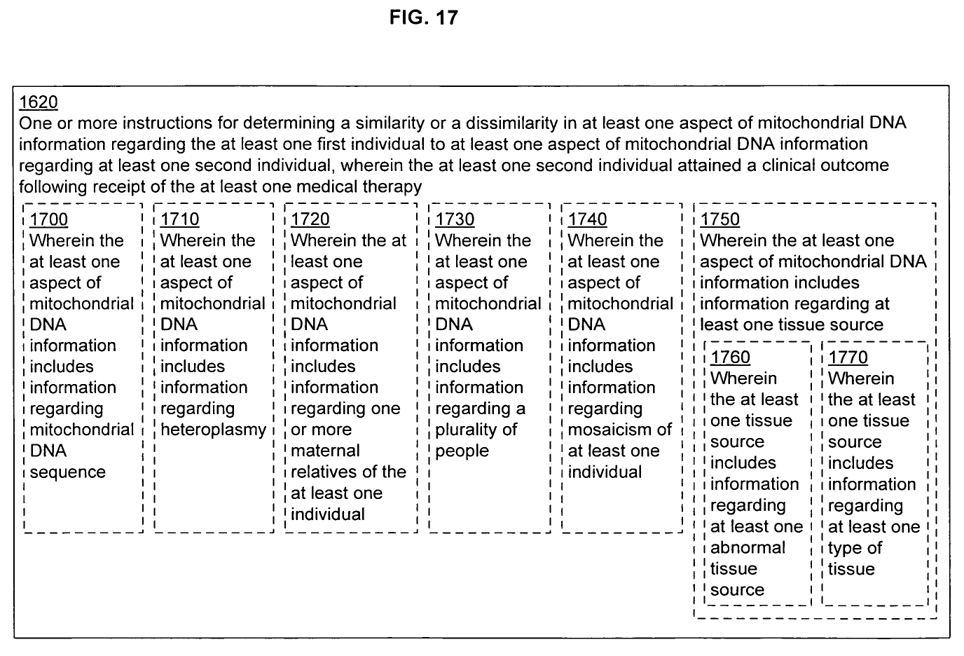

FIG. 17 illustrates aspects of a system such as that shown in FIG. 16. The one or more instructions for determining a similarity or a dissimilarity in at least one aspect of mitochondrial DNA information regarding the at least one first individual to at least one aspect of mitochondrial DNA information regarding at least one second individual, wherein the at least one second individual attained a clinical outcome following receipt of the at least one medical therapy 1620 may include one or more instructions wherein the at least one aspect of mitochondrial DNA information includes information regarding mitochondrial DNA sequence 1700. The one or more instructions for determining a similarity or a dissimilarity 1620 may include one or more instructions wherein the at least one aspect of mitochondrial DNA information includes information regarding heteroplasmy 1710. The one or more instructions for determining a similarity or a dissimilarity 1620 may include one or more instructions wherein the at least one aspect of mitochondrial DNA information includes information regarding one or more maternal relatives of the at least one individual 1720. The one or more instructions for determining a similarity or a dissimilarity 1620 may include one or more instructions wherein the at least one aspect of mitochondrial DNA information includes information regarding a plurality of people 1730. The one or more instructions for determining a similarity or a dissimilarity 1620 may include one or more instructions wherein the at least one aspect of mitochondrial DNA information includes information regarding mosaicism of at least one individual 1740. The one or more instructions for determining a similarity or a dissimilarity 1620 may include one or more instructions wherein the at least one aspect of mitochondrial DNA information includes information regarding at least one tissue source 1750. The one or more instructions where the at least one aspect of mitochondrial DNA information includes information regarding at least one tissue source 1750 may include one or more instructions wherein the at least one tissue source includes information regarding at least one abnormal tissue source 1760. The one or more instructions where the at least one aspect of mitochondrial DNA information includes information regarding at least one tissue source 1750 may include one or more instructions wherein the at least one tissue source includes information regarding at least one type of tissue 1770.

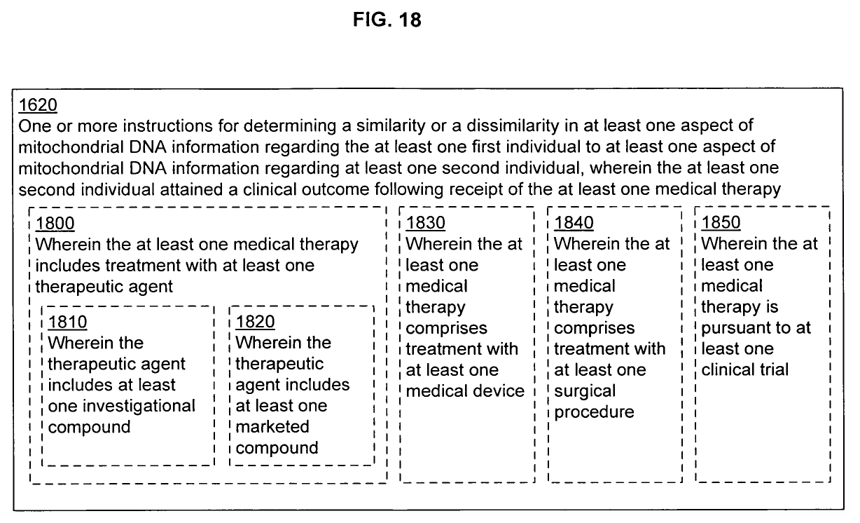

FIG. 18 depicts aspects of a system such as that shown in FIG. 16. The one or more instructions for determining a similarity or a dissimilarity in at least one aspect of mitochondrial DNA information regarding the at least one first individual to at least one aspect of mitochondrial DNA information regarding at least one second individual, wherein the at least one second individual attained a clinical outcome following receipt of the at least one medical therapy 1620 may include one or more instructions wherein the at least one medical therapy includes treatment with at least one therapeutic agent 1800. The one or more instructions wherein the at least one medical therapy includes treatment with at least one therapeutic agent 1800 may include one or more instructions wherein the therapeutic agent includes at least one investigational compound 1810. The one or more instructions wherein the at least one medical therapy includes treatment with at least one therapeutic agent 1800 may include one or more instructions wherein the therapeutic agent includes at least one marketed compound 1820. The one or more instructions for determining a similarity or a dissimilarity 1620 may include one or more instructions wherein the at least one medical therapy comprises treatment with at least one medical device 1830. The one or more instructions for determining a similarity or a dissimilarity 1620 may include one or more instructions wherein the at least one medical therapy comprises treatment with at least one surgical procedure 1840. The one or more instructions for determining a similarity or a dissimilarity 1620 may include one or more instructions wherein the at least one medical therapy is pursuant to at least one clinical trial 1850.

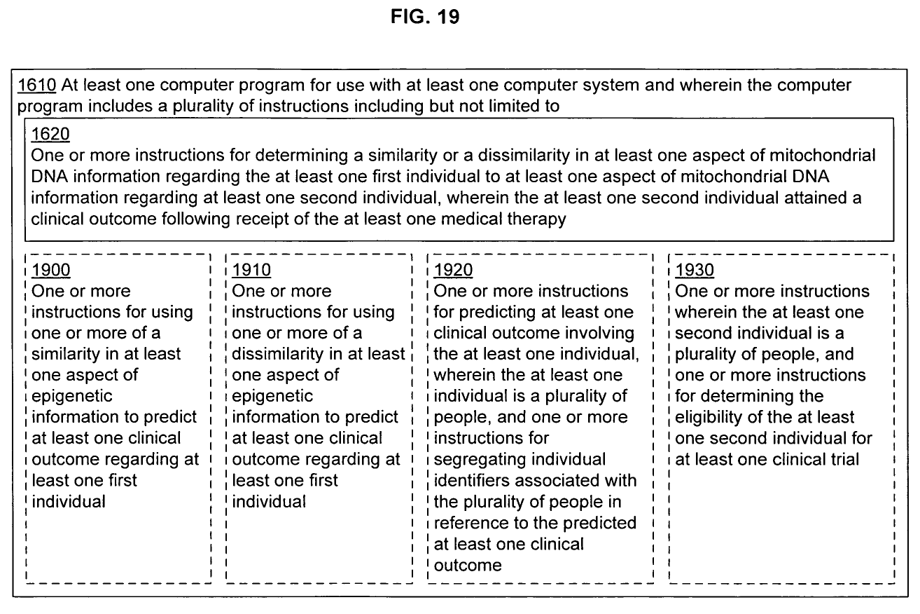

FIG. 19 shows aspects of a system such as that shown in FIG. 16. At least one computer program for use with at least one computer system 1610 may include one or more instructions for determining a similarity or a dissimilarity in at least one aspect of mitochondrial DNA information regarding the at least one first individual to at least one aspect of mitochondrial DNA information regarding at least one second individual, wherein the at least one second individual attained a clinical outcome following receipt of the at least one medical therapy 1620. At least one computer program for use with at least one computer system 1610 may include one or more instructions for using one or more of a similarity in at least one aspect of epigenetic information to predict at least one clinical outcome regarding at least one first individual 1900. At least one computer program for use with at least one computer system 1610 may include one or more instructions for using one or more of a dissimilarity in at least one aspect of epigenetic information to predict at least one clinical outcome regarding at least one first individual 1910. At least one computer program for use with at least one computer system 1610 may include one or more instructions for predicting at least one clinical outcome involving the at least one individual, wherein the at least one individual is a plurality of people, and one or more instructions for segregating individual identifiers associated with the plurality of people in reference to the predicted at least one clinical outcome 1920. At least one computer program for use with at least one computer system 1610 may include one or more instructions wherein the at least one individual is a plurality of people, and one or more instructions for determining the eligibility of the at least one second individual for at least one clinical trial 1930.

### B. Operation(s) or Process(es)

Following are a series of flowcharts depicting implementations of processes. For ease of understanding, the flowcharts are organized such that the initial flowcharts present implementations via an overall “big picture” or “top-level” viewpoint and thereafter the subsequent flowcharts present alternate implementations or expansions of the “big picture” flowcharts as either sub-steps or additional steps building on one or more earlier-presented flowcharts. Those having skill in the art will appreciate that the style of presentation utilized herein (e.g., beginning with a presentation of a flowchart(s) presenting an overall view and thereafter providing additions to or further details in subsequent flowcharts) generally allows for a more rapid and reliable understanding of the various process implementations.

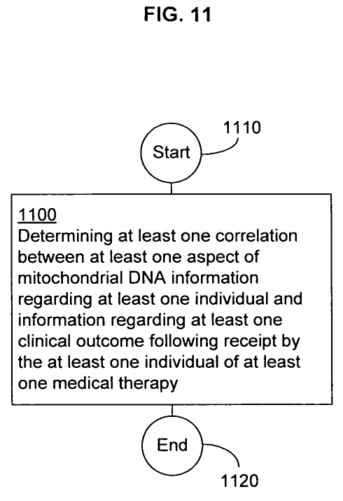

With reference now to FIG. 11, illustrated is a flowchart of a method. The method start is depicted at block 1110. Block 1100 depicts determining at least one correlation between at least one aspect of mitochondrial DNA information regarding at least one individual and information regarding at least one clinical outcome following receipt by the at least one individual of at least one medical therapy. Block 1120 depicts the end of the process.

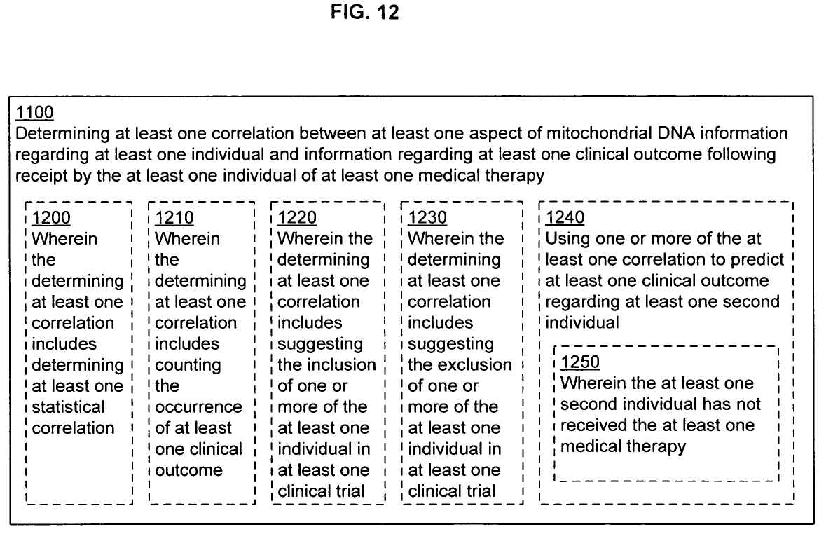

FIG. 12 shows aspects of the flowchart depicted in FIG. 11. Block 1100 depicts determining at least one correlation between at least one aspect of mitochondrial DNA information regarding at least one individual and information regarding at least one clinical outcome following receipt by the at least one individual of at least one medical therapy. Optional block 1200 depicts wherein the determining at least one correlation includes determining at least one statistical correlation. Optional block 1210 shows wherein the determining at least one correlation includes counting the occurrence of at least one clinical outcome. Optional block 1220 illustrates wherein the determining at least one correlation includes suggesting the inclusion of one or more of the at least one person in at least one clinical trial. Optional block 1230 depicts suggesting the exclusion of one or more of the at least one person in at least one clinical trial. Optional block 1240 depicts using one or more of the at least one correlation to predict at least one clinical outcome regarding at least one second individual. Optional block 1240 may include optional block 1250, illustrating wherein the at least one second individual has not received the at least one medical therapy.

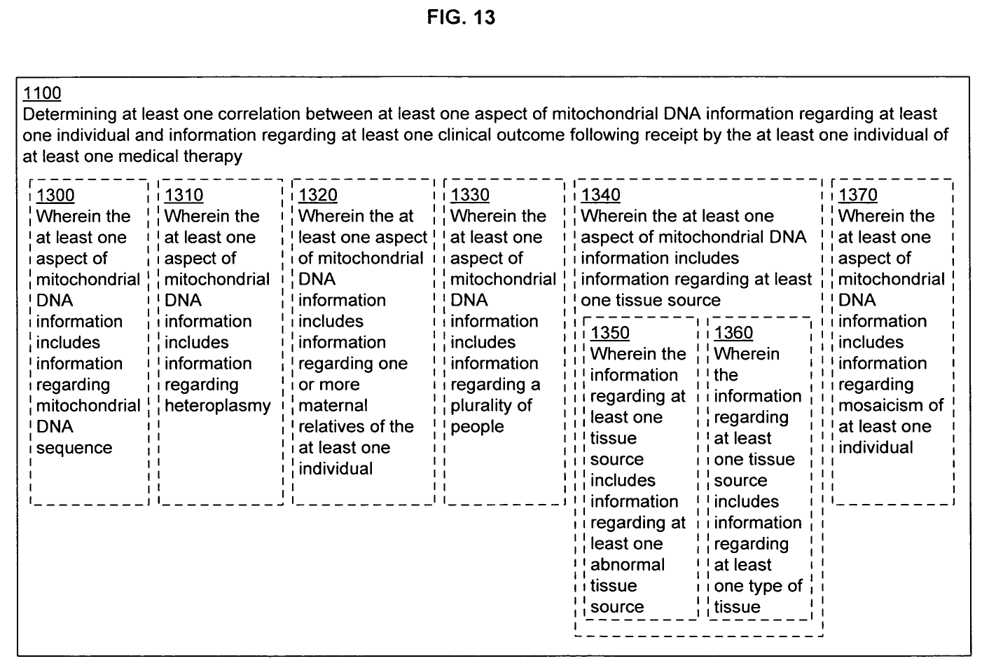

FIG. 13 illustrates aspects of the flowchart depicted in FIG. 11. Block 1100 depicts determining at least one correlation between at least one aspect of mitochondrial DNA information regarding at least one individual and information regarding at least one clinical outcome following receipt by the at least one individual of at least one medical therapy. Optional block 1300 shows wherein the at least one aspect of mitochondrial DNA information includes information regarding mitochondrial DNA sequence. Optional block 1310 illustrates wherein the at least one aspect of mitochondrial DNA information includes information regarding heteroplasmy. Optional block 1320 shows wherein the at least one aspect of mitochondrial DNA information includes information regarding one or more maternal relatives of the at least one individual. Optional block 1330 depicts wherein the at least one aspect of mitochondrial DNA information includes information regarding a plurality of people. Optional block 1340 illustrates wherein the at least one aspect of mitochondrial DNA information includes information regarding at least one tissue source. Optional block 1340 may include optional block 1350 depicting wherein the information regarding at least one tissue source includes information regarding at least one abnormal tissue source. Optional block 1340 may include optional block 1360 illustrating wherein the information regarding at least one tissue source includes information regarding at least one type of tissue. Optional block 1370 shows wherein the at least one aspect of mitochondrial DNA information includes information regarding mosaicism of least one individual.

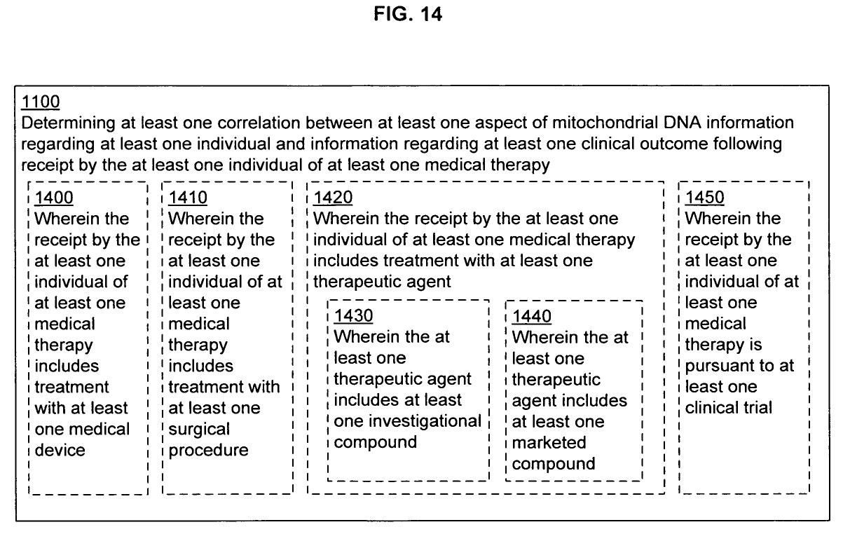

FIG. 14 illustrates aspects of the flowchart depicted in FIG. 11. Block 1100 depicts determining at least one correlation between at least one aspect of mitochondrial DNA information regarding at least one individual and information regarding at least one clinical outcome following receipt by the at least one individual of at least one medical therapy. Optional block 1400 depicts wherein the receipt by the at least one individual of at least one medical therapy includes treatment with at least one medical device. Optional block 1410 shows wherein the receipt by the at least one individual of at least one medical therapy includes treatment with at least one surgical procedure. Optional block 1420 illustrates wherein the receipt by the at least one individual of at least one medical therapy includes treatment with at least one therapeutic agent. Optional block 1420 may include optional block 1430, illustrating wherein the at least one therapeutic agent includes at least one investigational compound. Optional block 1420 may include optional block 1440, depicting wherein the at least one therapeutic agent includes at least one marketed compound. Optional block 1450 shows wherein the receipt by the at least one individual of at least one medical therapy is pursuant to at least one clinical trial.

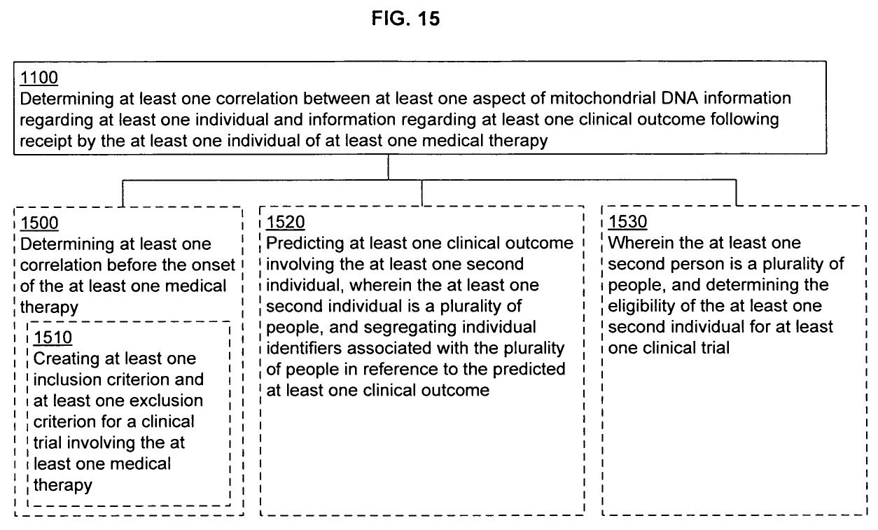

FIG. 15 shows aspects of the flowchart depicted in FIG. 11. Block 1100 depicts determining at least one correlation between at least one aspect of mitochondrial DNA information regarding at least one individual and information regarding at least one clinical outcome following receipt by the at least one individual of at least one medical therapy. Optional block 1500 illustrates determining at least one correlation before the onset of the at least one medical therapy. Optional block 1500 may include optional block 1510, depicting creating at least one inclusion criterion and at least one exclusion criterion for a clinical trial involving the at least one medical therapy. Optional block 1520 depicts predicting at least one clinical outcome involving the at least one second individual, wherein the at least one second individual is a plurality of people, and segregating individual identifiers associated with the plurality of people in reference to the predicted at least one clinical outcome. Optional block 1530 shows wherein the at least one second person is a plurality of people, and determining the eligibility of the at least one second individual for at least one clinical trial.

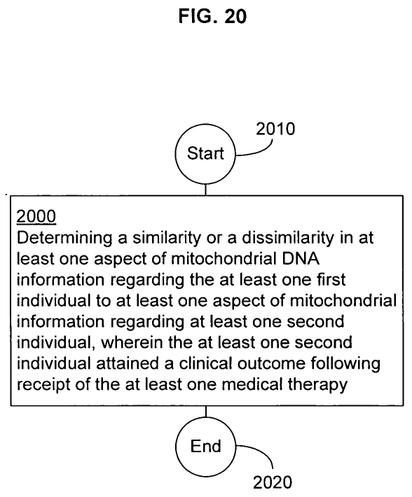

FIG. 20 depicts a flowchart of a method. The method start is depicted at block 2010. Block 2000 depicts determining a similarity or a dissimilarity in at least one aspect of mitochondrial DNA information regarding the at least one first individual to at least one aspect of mitochondrial information regarding at least one second individual, wherein the at least one second individual attained a clinical outcome following receipt of the at least one medical therapy. Block 2020 depicts the end of the process.

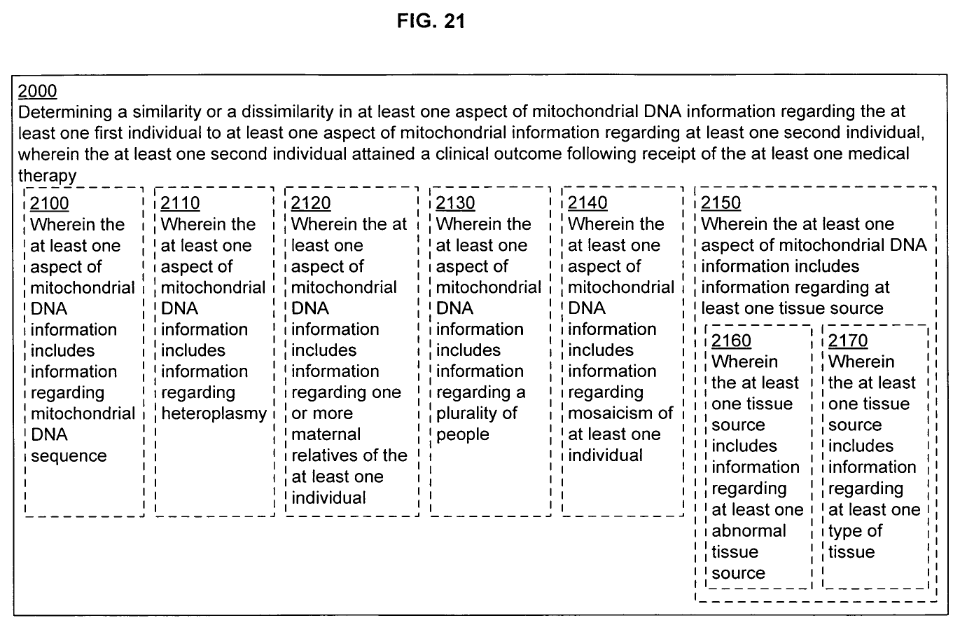

FIG. 21 illustrates aspects of the flowchart depicted in FIG. 20. Block 2000 may include one or more of optional blocks 2100, 2110, 2120, 2130, 2140, and 2150. Optional block 2150 may include one or more of optional blocks 2160 and 2170. Optional block 2100 depicts wherein the at least one aspect of mitochondrial DNA information includes information regarding mitochondrial DNA sequence. Optional block 2110 illustrates wherein the at least one aspect of mitochondrial DNA information includes information regarding heteroplasmy. Optional block 2120 shows wherein the at least one aspect of mitochondrial DNA information includes information regarding one or more maternal relatives of the at least one individual. Optional block 2130 depicts wherein the at least one aspect of mitochondrial DNA information includes information regarding a plurality of people. Optional block 2140 shows wherein the at least one aspect of mitochondrial DNA information includes information regarding mosaicism of at least one individual. Optional block 2150 illustrates wherein the at least one aspect of mitochondrial DNA information includes information regarding at least one tissue source 2150. Optional block 2150 may include optional block 2160, wherein the at least one tissue source includes information regarding at least one abnormal tissue source. Optional block 2150 may include optional block 2170, wherein the at least one tissue source includes information regarding at least one type of tissue.

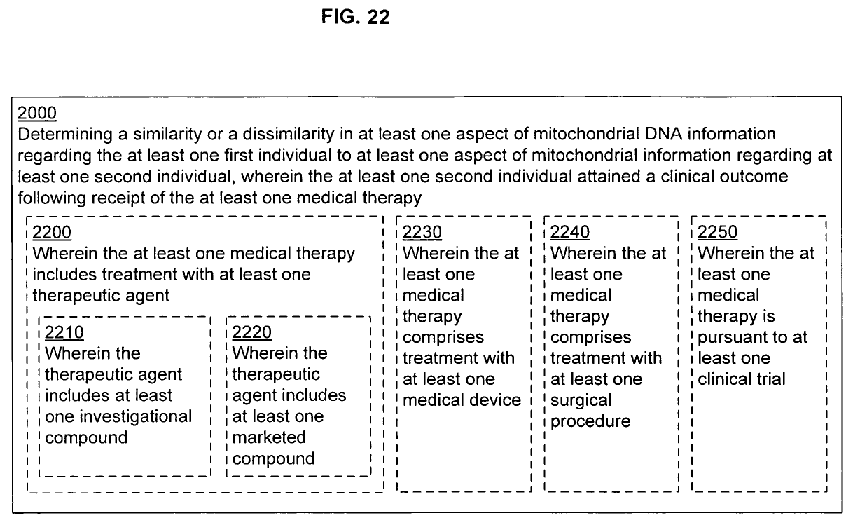

FIG. 22 illustrates aspects of the flowchart depicted in FIG. 20. Block 2000 may include one or more of optional blocks 2200, 2230, 2240, and 2250. Optional block 2200 may include one or more of optional blocks 2210 and 2220. Optional block 2200 depicts wherein the at least one medical therapy includes treatment with at least one therapeutic agent. Optional block 2200 may include optional block 2210 illustrating wherein the therapeutic agent includes at least one investigational compound. Optional block 2200 may include optional block 2220 depicting wherein the therapeutic agent includes at least one marketed compound. Optional block 2230 shows wherein the at least one medical therapy comprises treatment with at least one medical device. Optional block 2240 illustrates wherein the at least one medical therapy comprises treatment with at least one surgical procedure. Optional block 2250 depicts wherein the at least one medical therapy is pursuant to at least one clinical trial.

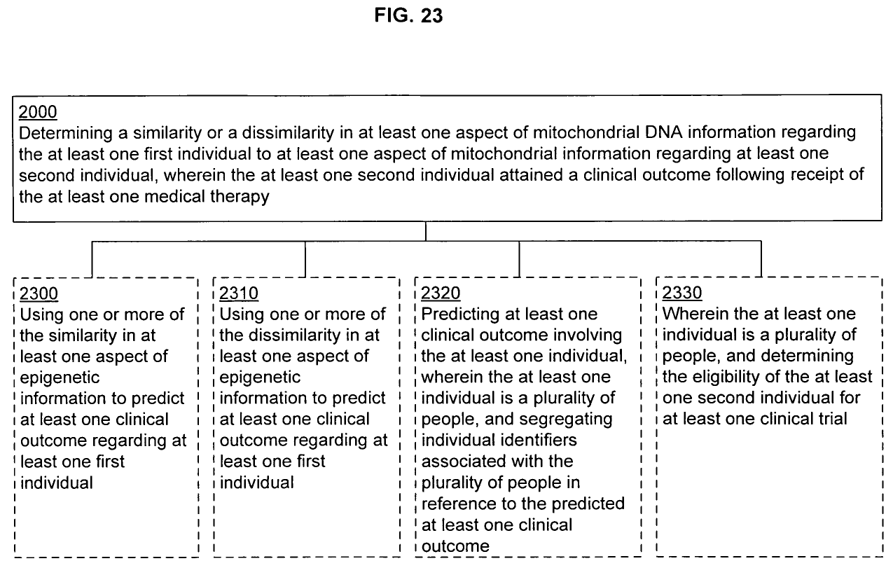

FIG. 23 depicts aspects of the flowchart depicted in FIG. 20. Block 2000 depicts determining a similarity or a dissimilarity in at least one aspect of mitochondrial DNA information regarding the at least one first individual to at least one aspect of mitochondrial information regarding at least one second individual, wherein the at least one second individual attained a clinical outcome following receipt of the at least one medical therapy. A flowchart may include one or more of optional blocks 2300, 2310, 2320, and 2330. Optional block 2300 illustrates using one or more of a similarity in at least one aspect of epigenetic information to predict at least one clinical outcome regarding at least one first individual. Optional block 2310 shows using one or more of a dissimilarity in at least one aspect of epigenetic information to predict at least one clinical outcome regarding at least one first individual. Optional block 2320 illustrates predicting at least one clinical outcome involving the at least one individual, wherein the at least one individual is a plurality of people, and segregating individual identifiers associated with the plurality of people in reference to the predicted at least one clinical outcome. Optional block 2330 shows wherein the at least one individual is a plurality of people, and determining the eligibility of the at least one second individual for at least one clinical trial.

### Variation(s), or Implementation(s)

Those having skill in the art will recognize that the present application teaches modifications of the devices, structures, or processes within the spirit of the teaching herein. For example, methods and systems described herein may be beneficial for correlating the at least one aspect of mitochondrial DNA information regarding at least one person with differential treatment response in a clinical or outpatient setting. The correlation between at least one aspect of mitochondrial DNA information regarding at least one person with positive or negative clinical outcome to a therapy, such as a therapeutic agent, investigational agent, energy-based therapy or surgical procedure, for example, may be used to identify the patient population that will derive the most benefit from the therapy.

The correlation between at least one aspect of mitochondrial DNA information regarding at least one person and a positive or negative clinical outcome to a therapy can be determined by prospective or retrospective data incorporated into the systems described herein. For example, data derived retrospectively from the scientific or medical literature may indicate that the presence or absence of one or more mitochondrial DNA variance correlates with a particular disease state. Mutations in human mitochondrial DNA increase with age and have been linked to various aspects of aging, such as sarcopenia or loss of muscle mass, Parkinson's and Alzheimer's diseases, ischemic heart disease, cataracts, and hearing loss, (see, e.g.: Kujoth et al., The role of mitochondrial DNA mutations in mammalian aging, PLOS Genetics, 3: e24 (2007); Wallace, Mitochondrial DNA sequence variation in human evolution and disease, PNAS USA 91:8739-8746 (1994); and Liu et al., Mutations in mitochondrial DNA accumulate differently in three different human tissues during aging, Nucleic Acids Research 26: 1268-1275 (1998), which are herein incorporated by reference). Data derived retrospectively from the scientific or medical literature may also indicate that the presence or absence of one or more mitochondrial DNA variance correlates with a positive or negative clinical outcome. As an example, sequence variations in the 12S and 16S rRNA of mitochondrial DNA predispose patients treated with aminoglycoside antibiotics to permanent hearing loss (Hutchin, et al., A molecular basis for human hypersensitivity to aminoglycoside antibiotics, Nucleic Acids Res. 21:4174-4179 (1993), which is herein incorporated by reference). As such, a diagnostic test or tests may be developed and used to determine at least one aspect of mitochondrial DNA information of a potential participant in a clinical trial, for example, prior to or as part of a clinical trial. Mitochondrial DNA information derived from a diagnostic test or tests may be incorporated, for example, as part of the inclusion/exclusion criteria used in segregating clinical trial participants and may identify the clinical trial participants for whom the therapy would provide the most positive clinical outcome.

Similarly, data derived retrospectively from the scientific or medical literature may indicate that the presence or absence of one or more mitochondrial DNA variance correlates with one or more negative clinical outcome. As such, a diagnostic test or tests may be incorporated into the clinical trial to monitor the development or progression of negative clinical outcomes correlated with person-specific mitochondrial DNA information. For example, if increased blood pressure is correlated with specific mitochondrial DNA information and a therapy, routine use of a blood pressure monitor may be incorporated into the clinical trial design. In some instances, a negative clinical outcome of a therapy correlated with specific mitochondrial DNA information may be progressive. For instance, individuals having certain mitochondrial DNA information may be prone to liver damage as a result a therapy. Based on this information, for example, one or more individuals may be excluded from the clinical trial. Alternatively, a diagnostic test may be incorporated into the clinical trial to routinely monitor, for example, liver enzymes as an indicator of potential liver damage. In some instances, a negative clinical outcome of a therapy correlated with specific aspects of mitochondrial DNA information may be life-threatening, in which case one or more individuals may be excluded from the clinical trial.

The correlation between one or more therapies and one or more aspects of mitochondrial DNA information regarding at least one individual may be determined retrospectively from individuals who have been previously treated with a particular therapy and for whom data are available regarding positive and negative clinical outcomes. Data regarding, for example, positive and negative clinical outcomes are routinely collected for each person participating in a clinical trial under guidelines regulated by, for example, the Food and Drug Administration (FDA) (see e.g. Food and Drug Administration: Compliance Program Guidance Manual, Chapter 48: Bioresearch Monitoring (bearing the date of Feb. 23, 2001), which is herein incorporated by reference). The methods and systems described herein may be used to derive correlations between historical clinical trial data and aspects of recently acquired mitochondrial DNA information from the participants. These correlates may be used, for example, to establish inclusion/exclusion criteria for future clinical trials or to guide prescribing practices.

Alternatively, data may be generated prospectively, for example, during the course of a clinical trial. As such, at least one aspect of mitochondrial DNA information may be collected for each participant prior to the initiation of the clinical trial. The methods and systems described herein may be used, for example, in real time to generate correlations between at least one aspect of mitochondrial DNA information regarding at least one individual and positive or negative clinical outcomes observed during the course of the clinical trial. It is anticipated that the methods and systems described herein may be used in conjunction with clinical data management systems, computerized or otherwise, for monitoring clinical data as regulated by, for example, the Food and Drug Administration (see e.g. Guidance for Industry: Computerized Systems Used in Clinical Investigations, Federal Registrar, 72:26638, May 10, 2007, which is herein incorporated by reference). As a clinical trial progresses, certain aspects of mitochondrial DNA information may correlate very early with substantial benefit to one or more subpopulations of participating individuals. The clinical trial sponsor may choose to use these correlations to modify the clinical trial design by altering, for example, the inclusion/exclusion criteria for future enrollment of participants. Similarly, as a clinical trial progresses, certain aspects of mitochondrial DNA information may correlate with the occurrence of one or more negative clinical outcome that may require additional monitoring of specific individuals, an event not originally planned for in the clinical trial design. The correlations determined during the clinical trial between therapy and at least one aspect of mitochondrial DNA information and clinical outcome may be used, for example, to design additional clinical trials with, for example, a narrowed patient population for whom the therapy will have the most positive and least negative clinical outcome.

The methods and systems described herein may be used as outlined above to develop a large body of correlative data regarding at least one aspect of mitochondrial DNA information of at least one individual and one or more medical therapies. In some embodiments, the at least one aspect of mitochondrial DNA information may include information from two or more individuals with a common attribute. At least one common attribute may include, for example, information regarding gender, height, weight, diabetes status, heart disease status, medical diagnosis, results on one or more medical tests, or ethnic background. Information regarding at least one tissue source may include information regarding the origin, storage, pathology, pathological subtype, or handling of the tissue and may include information regarding at least one neoplastic source, displastic source, diseased source, infectious source or cancerous source. At least one aspect of mitochondrial DNA information may also be correlated with pharmacogenetic information, such as polymorphisms in genes encoding enzymes associated with drug metabolism and transport (see e.g. Goldstein, et al., Pharmacogenetics goes genomic, Nature Rev. Genet. 4:937-947 (2003), which is herein incorporated by reference). The accumulated correlation data may be beneficial not only for clinical trial design and progression as described herein, but also for prescribing practices following approval of a new therapy. For example, a physician or other practitioner may use the accumulated correlation data in combination with the mitochondrial DNA information of an individual to predict whether a specific medical therapy will provide a positive outcome. In addition, a physician or other practitioner may use the accumulated correlation data to predict specific negative outcomes that may be associated with a given treatment and at least one aspect of an individual's specific mitochondrial DNA information. As such, the physician or person may use these data to assess the risk/benefit associated with a particular treatment option and make decisions regarding treatment accordingly. In the instance, for example, where there are multiple therapies of a given class available to treat a specific disease or condition, the physician or other practitioner may use the accumulated correlation data for each therapy in combination with at least one aspect of the person's specific mitochondrial DNA information to choose the optimal treatment course.

The methods and systems described herein may also be beneficial for predicting potential disease outcome and may aid a physician or other practitioner in developing appropriate treatment options. For example, a series of five primary mutations in mitochondrial DNA have been linked to Leber hereditary optic neuropathy (LHON), a maternally inherited form of late-onset vision loss (Wallace, Mitochondrial DNA sequence variation in human evolution and disease, Proc. Natl. Acad. Sci. 91:8739-8746 (1994), which is herein incorporated by reference). Two of the most severe LHON mutations are also associated with pediatric neurodegenerative disease, suggesting that the milder LHON mutations may predispose individuals to late-onset neurodegenerative diseases, such as Parkinson's and Alzheimer's diseases. In this example, the physician or other practitioner may use at least one aspect of an individual's mitochondrial DNA information regarding LHON mutations to predict patient outcome and to determine appropriate treatment options, such as more frequent visits to the opthalmologist and early tests for neurodegenerative disease.

The methods and systems described herein may also be beneficial in assessing the potential toxicity of one or more investigational or existing therapies during clinical trial progression or in an outpatient setting. For example, at least one aspect of the mitochondrial DNA information of an individual may be determined prior to initiation of a clinical trial and the data incorporated into a database accessible by the system described herein. As the clinical trial proceeds, at least one aspect of mitochondrial DNA information may be periodically reassessed and monitored for any changes. Changes in aspects of the mitochondrial DNA information may or may not be correlated with one or more negative clinical outcome observed during the time course of the study. Any changes in aspects of the mitochondrial DNA information may be linked back to known information regarding specific mitochondrial DNA changes and possible outcomes. For example, nucleoside analogs used as antiviral therapy for human immunodeficiency virus (HIV) and hepatitis B are known to induce mutations in mitochondrial DNA as well as perturb mitochondrial DNA synthesis as measured by a decrease in overall mitochondrial DNA (see e.g. Martin, et al., Accumulation of mitochondrial DNA mutations in human immunodeficiency virus-infected patients treated with nucleoside analogue reverse-transcriptase inhibitors, Am. J. Hum. Genet. 72:549-560 (2003); and Johnson, et al., Toxicity of antiviral nucleoside analogs and the human mitochondrial DNA polymerase, J. Biol. Chem. 276:40847-40857 (2001), which are herein incorporated by reference). Several of the negative clinical outcomes associated with antiviral treatment, including lipoatrophy and neuropathy, mimic the symptoms associated with genetic mutations in mitochondrial DNA. As such, the Food and Drug Administration has recommended that all investigational antiviral agents be tested for effects on mitochondrial DNA (see e.g. Guidance for industry: antiviral product development—conducting and submitting virology studies to the agency, Food and Drug Administration, bearing the date of June 2006, which is herein incorporated by reference). More generally, one or more changes in aspects of the mitochondrial DNA information over the course of treatment with, for example, an investigational agent, may indicate increased risk of developing a disease in the future. In addition, changes in aspects of the mitochondrial DNA information associated with a specific investigational agent can be compared, for example, with one or more agents with comparable method of action to determine if changes in at least one aspect of mitochondrial DNA information is a class phenomenon associated with all agents possessing a similar method of action or specific to the chemical entity under investigation.

Other modifications of the subject matter herein will be appreciated by one of ordinary skill in the art in light of the teachings herein.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes or systems or other technologies described herein can be effected (e.g., hardware, software, or firmware), and that the preferred vehicle will vary with the context in which the processes or systems or other technologies are deployed. For example, if an implementer determines that speed and accuracy are paramount, the implementer may opt for a mainly hardware or firmware vehicle; alternatively, if flexibility is paramount, the implementer may opt for a mainly software implementation; or, yet again alternatively, the implementer may opt for some combination of hardware, software, or firmware. Hence, there are several possible vehicles by which the processes or devices or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In a general sense, those skilled in the art will recognize that the various aspects described herein which can be implemented, individually or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes or devices described herein, or a microprocessor configured by a computer program which at least partially carries out processes or devices described herein), electrical circuitry forming a memory device (e.g., forms of random access memory), or electrical circuitry forming a communications device (e.g., a modem, communications switch, or optical-electrical equipment). Those having skill in the art will recognize that the subject matter described herein may be implemented in an analog or digital fashion or some combination thereof.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, those skilled in the art will appreciate that recited operations therein may generally be performed in any order. Examples of such alternate orderings may include overlapping, interleaved, interrupted, reordered, incremental, preparatory, supplemental, simultaneous, reverse, or other variant orderings, unless context dictates otherwise. With respect to context, even terms like “responsive to,” “related to”, or other past-tense adjectives are generally not intended to exclude such variants, unless context dictates otherwise.

With respect to the use of substantially any plural or singular terms herein, those having skill in the art can translate from the plural to the singular or from the singular to the plural as is appropriate to the context or application. The various singular/plural permutations are not expressly set forth herein for sake of clarity.

All of the above U.S. patents, U.S. patent application publications, U.S. patent applications, foreign patents, foreign patent applications and non-patent publications referred to in this specification or listed in any Application Data Sheet, are incorporated herein by reference, to the extent not inconsistent herewith.

The foregoing detailed description has set forth various embodiments of the devices or processes via the use of block diagrams, flowcharts, or examples. Insofar as such block diagrams, flowcharts, or examples contain one or more functions or operations, it will be understood by those within the art that each function or operation within such block diagrams, flowcharts, or examples can be implemented, individually or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital or an analog communication medium (e.g., a fiber optic cable, a waveguide, a wired communications link, a wireless communication link, etc.).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method comprising:
determining at least one correlation between at least one aspect of mitochondrial DNA information regarding at least one individual and information regarding at least one clinical outcome following receipt by the at least one individual of at least one medical therapy.

2. The method of claim 1, wherein the determining at least one correlation further comprises:
determining at least one statistical correlation.

3. The method of claim 1, wherein the determining at least one correlation further comprises:
counting the occurrence of at least one clinical outcome.

4. The method of claim 1, wherein the at least one aspect of mitochondrial DNA information further comprises:
information regarding mitochondrial DNA sequence.

5. The method of claim 1, wherein the at least one aspect of mitochondrial DNA information further comprises:
information regarding heteroplasmy.

6. The method of claim 1, wherein at least one aspect of mitochondrial DNA information further comprises:
information regarding one or more maternal relatives of the at least one individual.

7. (canceled)

8. The method of claim 1, wherein the at least one aspect of mitochondrial DNA information further comprises:
information regarding at least one tissue source.

9. (canceled)

10. (canceled)

11. The method of claim 1, wherein the at least one aspect of mitochondrial DNA information further comprises:
information regarding mosaicism of at least one individual.

12. (canceled)

13. (canceled)

14. The method of claim 1, wherein the receipt by the at least one individual of at least one medical therapy includes treatment with at least one therapeutic agent.

15. (canceled)

16. (canceled)

17. The method of claim 1, wherein the receipt by the at least one individual of at least one medical therapy is pursuant to at least one clinical trial.

18. (canceled)

19. The method of claim 18, further comprising:
creating at least one inclusion criterion and at least one exclusion criterion for a clinical trial involving the at least one medical therapy.

20. (canceled)

21. (canceled)

22. The method of claim 1, further comprising:
using one or more of the at least one correlation to predict at least one clinical outcome regarding at least one second individual.

23. (canceled)

24. (canceled)

25. The method of claim 1, further comprising:
wherein the at least one second individual is a plurality of people; and
determining the eligibility of the at least one second individual for at least one clinical trial.

26. A method of predicting a clinical outcome of at least one medical therapy for at least one first individual, comprising:
determining a similarity or a dissimilarity in at least one aspect of mitochondrial DNA information regarding the at least one first individual to at least one aspect of mitochondrial information regarding at least one second individual, wherein the at least one second individual attained a clinical outcome following receipt of the at least one medical therapy.

27. The method of claim 26, wherein the at least one aspect of mitochondrial DNA information further comprises:
information regarding mitochondrial DNA sequence.

28. The method of claim 26, wherein the at least one aspect of mitochondrial DNA information further comprises:
information regarding heteroplasmy.

29. The method of claim 26, wherein at least one aspect of mitochondrial DNA information further comprises:
information regarding one or more maternal relatives of the at least one individual.

30. (canceled)

31. The method of claim 26, wherein the at least one aspect of mitochondrial DNA information further comprises:
information regarding at least one tissue source.

32. (canceled)

33. (canceled)

34. The method of claim 26, wherein the at least one aspect of mitochondrial DNA information further comprises:
information regarding mosaicism of at least one individual.

35. (canceled)

36. (canceled)

37. The method of claim 26, wherein the at least one medical therapy includes treatment with at least one therapeutic agent.

38. (canceled)

39. (canceled)

40. The method of claim 26, wherein the at least one medical therapy is pursuant to at least one clinical trial.

41. The method of claim 26, further comprising:
using one or more of the similarity in at least one aspect of epigenetic information to predict at least one clinical outcome regarding at least one first individual.

42. The method of claim 26, further comprising:
using one or more of the dissimilarity in at least one aspect of epigenetic information to predict at least one clinical outcome regarding at least one first individual.

43. (canceled)

44. The method of claim 26, further comprising:
wherein the at least one individual is a plurality of people; and
determining the eligibility of the at least one second individual for at least one clinical trial.

45. A system comprising:
at least one computer program for use with at least one computer system and wherein the computer program includes a plurality of instructions including but not limited to:
one or more instructions for determining at least one correlation between at least one aspect of mitochondrial DNA information regarding at least one individual and information regarding at least one clinical outcome following receipt by the at least one individual of at least one medical therapy.

46. The system of claim 45, wherein the one or more instructions for determining at least one correlation further comprises:
one or more instructions for determining at least one statistical correlation.

47. The system of claim 45, wherein the one or more instructions for determining at least one correlation further comprises:
one or more instructions for counting the occurrence of at least one clinical outcome.

48. The system of claim 45, wherein the at least one aspect of mitochondrial DNA information further comprises:
information regarding mitochondrial DNA sequence.

49. The system of claim 45, wherein the at least one aspect of mitochondrial DNA information further comprises:
information regarding heteroplasmy.

50. The system of claim 45, wherein the at least one aspect of mitochondrial DNA information further comprises:
information regarding one or more maternal relatives of the at least one individual.

51. The system of claim 45, wherein the at least one aspect of mitochondrial DNA information further comprises:
information regarding a plurality of people.

52. The system of claim 45, wherein the at least one aspect of mitochondrial DNA information further comprises:
information regarding at least one tissue source.

53. (canceled)

54. (canceled)

55. The system of claim 45, wherein the at least one aspect of mitochondrial DNA information further comprises:
information regarding mosaicism of at least one individual.

56. The system of claim 45, wherein the receipt by at least one individual of at least one medical therapy comprises treatment with at least one medical device.

57. The system of claim 45, wherein the receipt by at least one individual of at least one medical therapy comprises treatment with at least one surgical procedure.

58. The system of claim 45, wherein the receipt by at least one individual of at least one medical therapy comprises treatment with at least one therapeutic agent.

59. (canceled)

60. (canceled)

61. The system of claim 45, wherein the receipt by at least one individual of at least one medical therapy is pursuant to at least one clinical trial.

62. The system of claim 45, wherein the one or more instructions for determining at least one correlation further comprises:
one or more instructions for determining at least one correlation before the onset of the at least one medical therapy.

63. The system of claim 62, further comprising:
one or more instructions for creating at least one inclusion criterion and at least one exclusion criterion for a clinical trial involving at least one medical therapy.

64. (canceled)

65. (canceled)

66. The system of claim 45, further comprising:
one or more instructions for using one or more of the at least one correlation to predict at least one clinical outcome regarding at least one second individual.

67. The system of claim 66, wherein the at least one second individual has not received the at least one medical therapy.

68. (canceled)

69. The system of claim 45, further comprising:
one or more instructions wherein the at least one second individual is a plurality of people; and
one or more instructions for determining the eligibility of the at least one second individual for at least one clinical trial.

70. A system for predicting a clinical outcome of at least one medical therapy for at least one first individual, comprising:
at least one computer program for use with at least one computer system and wherein the computer program includes a plurality of instructions including but not limited to:
one or more instructions for determining a similarity or a dissimilarity in at least one aspect of mitochondrial DNA information regarding the at least one first individual to at least one aspect of mitochondrial DNA information regarding at least one second individual, wherein the at least one second individual attained a clinical outcome following receipt of the at least one medical therapy.

71. The system of claim 70, wherein the at least one aspect of mitochondrial DNA information further comprises:
information regarding mitochondrial DNA sequence.

72. The system of claim 70, wherein the at least one aspect of mitochondrial DNA information further comprises:
information regarding heteroplasmy.

73. The system of claim 70, wherein the at least one aspect of mitochondrial DNA information further comprises:
information regarding one or more maternal relatives of the at least one individual.

74. (canceled)

75. The system of claim 70, wherein the at least one aspect of mitochondrial DNA information further comprises:
information regarding at least one tissue source.

76. (canceled)

77. (canceled)

78. The system of claim 70, wherein the at least one aspect of mitochondrial DNA information further comprises:
information regarding mosaicism of at least one individual.

79. The system of claim 70, wherein the at least one medical therapy comprises treatment with at least one medical device.

80. The system of claim 70, wherein the at least one medical therapy comprises treatment with at least one surgical procedure.

81. The system of claim 70, wherein the at least one medical therapy comprises treatment with at least one therapeutic agent.

82. (canceled)

83. (canceled)

84. The system of claim 70, wherein the at least one medical therapy is pursuant to at least one clinical trial.

85. The system of claim 70, further comprising:
one or more instructions for using one or more of a similarity in at least one aspect of epigenetic information to predict at least one clinical outcome regarding at least one first individual.

86. The system of claim 70, further comprising:
one or more instructions for using one or more of a dissimilarity in at least one aspect of epigenetic information to predict at least one clinical outcome regarding at least one first individual.

87. (canceled)

88. The system of claim 70, further comprising:
one or more instructions wherein the at least one second individual is a plurality of people; and
one or more instructions for determining the eligibility of the at least one second individual for at least one clinical trial.

89. A system comprising:
at least one computer program for use with at least one computer system and wherein the computer program includes a plurality of instructions including but not limited to:
one or more instructions for making one or more correlations between at least one aspect of mitochondrial DNA information obtained regarding at least one individual and information regarding at least one medical therapy involving the at least one individual; and
one or more instructions for applying one or more of the one or more correlations to at least one aspect of mitochondrial DNA information obtained regarding a plurality of people.

90. The system of claim 89, further comprising:
one or more instructions for segregating individual identifiers associated with the plurality of people in reference to at least one of the one or more applied correlations.

91. The system of claim 89, wherein the at least one medical therapy includes at least one investigational therapeutic agent.

92. The system of claim 89, wherein the at least one aspect of mitochondrial DNA information includes mitochondrial DNA sequence information.

93. The system of claim 89, wherein the at least one aspect of mitochondrial DNA information includes information from two or more individuals with a common attribute.

94. (canceled)

95. The system of claim 89, wherein the at least one aspect of mitochondrial DNA information includes information regarding at least one tissue source.

96. The system of claim 89, further comprising:
one or more instructions for segregating individual identifiers associated with the plurality of people in reference to at least one characteristic shared by two or more individuals of the plurality of people.
